(19) United States
(12) Patent Application Publication
(10) Pub. No.: US 2003/0100493 A1
(43) Pub. Date: May 29, 2003

(54) Sublingual use of inhibitors in the biosynthesis of cholesterol

(76) Inventor: Sol Weiss, Reseda, CA (US)

Correspondence Address: MITCHELL, SILBERBERG & KNUPP, LLP, Trident Center, 11377 West Olympic Boulevard, Los Angeles, CA, 90064, US

(21) Appl. No.: 10/160,441

(22) Filed: Jun. 4, 2002

Related U.S. Application Data
(60) Provisional application No. 60/306,977, filed on Jul. 19, 2001.
(60) Provisional application No. 60/314,532, filed on Aug. 23, 2001.

(51) Int. Cl. A61K 38/17; A61K 31/56; A61K 31/401; A61K 31/366; A61K 31/225

(52) U.S. Cl. 514/12; 514/171; 514/423; 514/356; 514/460; 514/548

(57) ABSTRACT

The present invention is a method introducing the sublingual placement of statin drugs whose names include: Fluvastatin, Atorvastatin, Lovastatin, Pravastatin and Simvastatin for heart related and other vascular emergencies. Current research challenges are developing many new derivatives and new classes of these HMG-CoA reductase inhibitors, which alter the biosynthesis of cholesterol. This method applies these medications (statin drugs) in a form such as sublingual (under the tongue) for rapid absorption and immediate high blood levels similar to that of nitroglycerin. The advantage of this method is that it will benefit those who are stricken with strokes and heart attacks by therefore saving lives and costs of medical care.

### CROSS-REFERENCE TO RELATED APPLICATIONS

This application contains subject matter of my provisional application Serial No. 60/306,977, filed Jul. 19, 2001, entitled Sublingual Use of Inhibitors in the Biosynthesis of Cholesterol, and in my provisional application Serial No. 60/314,532, filed Aug. 23, 2001, entitled Amendments to Serial No. 60/306,977.

### BACKGROUND OF INVENTION

1. Field of Invention:

There is disclosed a method and combination of components to administer statin drugs in a beneficial method and manner. This method includes the sublingual administration of drugs selected from the class of drugs known as statin drugs. In addition to the method, there is disclosed a method and attendant components for administering sublingual medication or medications for emergency stabilization of ruptured plaques; suppression of thrombus or aggregation of platelets; alteration of inflammatory responses; improvement of endothelial function; reduction in cell death and augmentation of vasodilation. Disclosed is the combination of medications, such as a statin drug and nitroglycerin, and others, in sublingual administration. In addition, it has been found that combinations of other statin class drugs with other cholesterol lowering drugs such as niacin and other complications rupture, thrombus and platelet aggregation, inflammation, compromised endothelial function, vasoconstriction and cell death. These statin medications both by their own merits and in combinations with other drugs will lend to more beneficial and/or synergistic effects. Statins are like the new aspirins for use in the field of emergency medicine. The new Heart Protection Study (HPS) done in England reflects this latest and major breakthrough study of the statin class of drugs showing a reduction of adverse major vascular events over 5.5 years of treatment in high-risk patients. Most everyone is aware of the beneficial role of aspirin in emergency medicine.

Furthering this theory of use ill medical emergencies are new reports of statins reducing infarct size in stroke patients. Statins favor endothelial nitric oxide synthase (eNOS) and block inducible nitric oxide synthase (iNOS) effects. These effects are neuroprotective by preserving blood flow and limiting neurological insult. Another ongoing study shows that a neurotoxin, Beta-Amyloid, derived from the Amyloid Precursor Protein (APP), was found embedded in the membranes of cells as disclosed in Alzheimer's disease, multiple sclerosis, vascular inflammations and other degenerative changes. Similar changes are recognized in patients with coronary artery and vascular diseases therefore implicating the plaque deposits occur elsewhere in the body as a result of the high cholesterol contents in tissues. High cholesterol induces these toxic changes.

Other data show that statin drugs that do not pass the blood brain barrier such as Atorvastatin, Pravastatin and Fluvastatin are not associated with findings of increased cholesterol in the brain tissue that would be toxic.

The invention of the sublingual administration of HMG-Co reductase enzyme inhibitors and its analogues, derivatives, subparts or synthesized inhibitors and cholesterol ester transfer protein (CETP) pathway accelerator inhibitors and its analogues, derivatives, subparts or synthesized inhibitors referred herein as the “statin” medications will under the present invention be used for inhibiting cholesterol synthesis and hypertriglyceridemia among the most important advantages. The other advantages can alter the outcomes that occur in unstabilization of cholesterol plaques and the increased thromboembolic events that lead to heart attacks and strokes, a life saving event. These statins may be used in conjunction with other medications such as niacin and platelet aggregation inhibitors to alter the early onset of strokes, heart attack and other diseases.

2. Belated Art

In the 1960's, Congress passed laws requiring the development of emergency medical services. In 1968, the American College of Emergency Physicians (ACEP) was formed and by 1979 the American Board of Medical Specialties recognition to emergency medicine as the nation's 23rd medical specialty. Novel innovations and developments grew as a result, providing a new significant resource for the medical community. Newer applications of technology grew with this specialty as well.

In the past 30-40 years, the established treatment for coronary conditions dealt with physics and pathophysiology of the atherosclerotic processes that cause stenosis, or narrowing of the blood vessels, produce coronary events that are proportional to the amount of the stenosis. Increased myocardial oxygen demand is the exciting factor for this coronary event. More recently, it has been demonstrated that these mechanisms, which are directed at supply and demand and were treated, in its best approach with beta-blockers, vasodilators, by-pass surgeries and angioplasties, were found to fail in a number of patients. Heart attacks recurred and there remained no further explanations. The newer explanations now require attention to the biochemistry and less on the physics of the problem.

The new concepts rely on the following premises:

A. The cholesterol plaque becomes unstable, disrupts and results in thrombosis and/or emboli cause the acute event.

B. The tendency for rupture of the cholesterol plaque is less related to the stenosis severity than its quieter tendency to rely upon a softer and unstable plaque formation that is today undetectable. Treatment has to be directed at plaque stability. See Tables I and II.

C. People who have insulin resistance in tissues as seen in various clinical conditions such as syndrome X (shown below) have a greater propensity for plaque disruption. Any of the clinical examples may lead to plaque disruption and thrombosis.

Syndrome X includes: Obesity, hypertension, hypertriglyceridemia, increased small density LDL, low HDL, hypercoagulability and elevated microalbuminuria.

Recently, researchers in cardiovascular medicine at Ohio State University presented findings suggesting deficiencies in two-dimensional projections of lumen sizes with angiographic visualizations. The new evidence using intravascular ultrasound (IVUS) shows precise quantification of atherosclerotic disease. The tomographic orientation of ultrasound reveals a picture of the full 360-degree circumference of the vessel wall.

The IVUS greatly improves the visualization of diseased segments of vessels and characterizations of ostial stenosis, lesions at bifurcation sites and highly eccentric plaques.

Angiography is misled by a process of coronary “remodeling”, first described in 1987 by S. Glagov et al in the New England Journal of Medicine. This remodeling effect in blood vessels was noted as an outward displacement of the external vessel wall. This adventitial enlargement avoids the luminal or vascular circumference concealing the atheroma disease. These lesions do not necessarily obstruct the blood flow in their early disease process and will not offer the diagnostic evidence for evaluation of acute coronary syndromes and myocardial injury. These concealed events of the disrupted plaques escape detection with angiographic techniques, but not so with IVUS.

Therefore, the treatment must be aimed at stabilizing the plaques thrombus formations. The following are the effects of the “statin” drugs:

1. Lowers the lipid (fat) levels directly. See Table III.

2. Improves the endothelial membrane lining of the blood vessels to allow vessel dilation.

3. Inhibits platelet thrombus or aggregation causing occlusion and/or thromboembolic phenomena.

4. Stabilizes atherosclerotic plaque instability.

a) Lowers lipid content of the plaque. See Table III.

b) Lowers oxidized LDL. See Table II.

c) Lowers number of macrophages. See Table II.

d) Lowers level of T-cells.

e) Lowers Protenase enzymes that breakdown the plaque.

f) Lowers cell death (apoptosis).

g) Increases collengenase that strengthens the plaque.

h) Lowers the inflammatory changes in the plaque.

i) Reduces infarct sizes.

j) Neuroprotective by limiting neurological loss.

k) Reduces the number of strokes and vascular events.

The current testing of the “statins” in the industry involves the use of intravenous administration of Rosuvastatin, a significantly higher potency as an inhibitor of cholesterol synthesis than the other statins. It was suggested that Rosuvastatin has the potential to exert a profound effect on atherogenic lipoproteins. This also suggests that earlier, aggressive and higher dosing of statins can alter the equation in linear form, halting an ongoing complication in the above events, and thereby, preventing the progressing complications of plaque disruption and thromboembolic events. These are potent creative therapies particularly for the emergency situations. Recent tests show plaque stabilization as early as eighteen (18) hours with less aggressive therapy. The statin administration in a sublingual form with higher and immediate pre-hospital dosage should improve changes in outcomes and result in the:

1. Reduction in coronary events and their subsequent attacks.

2. Reduction in thrombus and aggregation of platelet formations. This suppression lessens occlusions and emboli, which devastate tissues such as seen in strokes, coronaries and amputations.

3. Increased dilation of arteries and better blood flow.

Transfers of the pre-hospital patients may have long delays. Benefits from earlier treatment, i.e. by sublingual dosing and nitroglycerin can be lifesaving. Another benefit of successful earlier treatment is that of preventing complications.

The recognized cardioprotective mechanism for HDL-cholesterol and its analogues, derivatives, subparts and synthesized inhibitors act as a vehicle for reverse cholesterol transport from tissue to liver. The level of HDL-cholesterol (the good cholesterol) is inversely correlated with the risk of coronary heart disease. ApoA-I and ApoA-II are the largest proteins of HDL-cholesterol. ApoA-I is felt to be the most important for reverse cholesterol transport and protection effects. See Table III. Protective effects may also include inhibition of smooth interference with macrocytosis. A combination of HDL-cholesterol (natural, derived and/or synthesized, subparts or analogues of HDL-cholesterol as a natural and non-natural protector and antioxidant) and a statin drug may offer a synergistic effect to moderate, neutralize and oppose the cholesterol atherogenetic pathogenesis.

### Cholesterol Synthesis

TABLE I

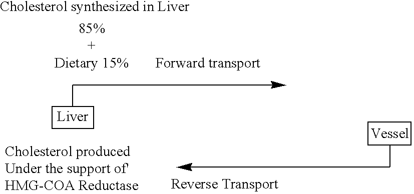

TABLE II

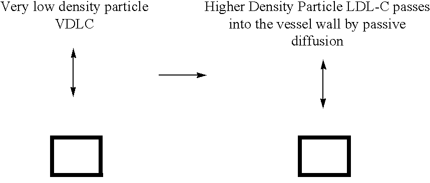

HMG-CoA
Reductase Enzyme
(statins are inhibitors)
Once inside the vessel wall, LDL undergoes an oxidative process.
Oxidized LDL is taken up by macrophages thereby converting this into
foam cells the early manifestation of the atheromatous process.
This is an inflammatory process.

### Role of Lipid Metabolism in Cholesterol Transport

TABLE III

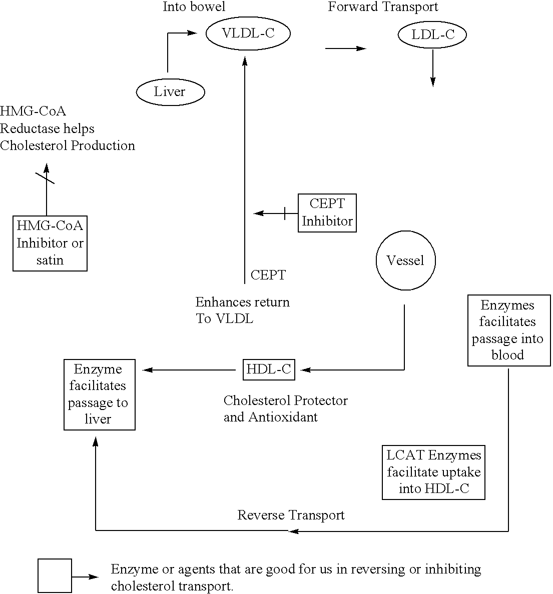

Insulin resistance accentuates forward cholesterol
transport, i.e.: Syndrome X
The Reverse Transport diagram indicates potential new medication
areas that will benefit patients in vascular diseases

### SUMMARY OF THE INVENTION

The sublingual administration of the present invention provides a method of rapid absorption as seen with sublingual nitroglycerin treatment of coronaries. Procardia, when administered in emergency situations sublingually to patients with severe hypertension, produces an immediate lowering of blood pressure. Another example is that of sublingual Levsin, which takes approximately nine (9) minutes before being completely absorbed, reduces acute smooth muscle spasms.

Therefore, the advantage of sublingual dosing are:

1) Rapid absorption into the blood for immediate tissue action.

2) High blood levels of medications can be achieved quickly as the absorption is not altered appreciably by the acid, alkalinity, enzymes, bile salts, fermentation, bacteria, inflammations and other diseases within the gastrointestinal tract.

3) Nausea and/or vomiting are not factors in blocking the administration of the drug.

4) Sublingual administration of nitroglycerin either together or in close timing of each other so that a coronary patient can receive the synergistic effects of both medications. The “statins” affect the paradoxical endothelial response of the blood vessels so that vasodilation is favored over the non-treated statin patients whose response is more vasoconstrictive. The nitroglycerin produces a short lasting effect (five to ten minutes). The statin effect is longer, thereby aiding in the vasodilation time for the patient. This will further aid the blood flow and the oxygenation of the tissues in those cases that would use both the nitroglycerin and the statin drugs together.

5) Three of the statin drugs are water-soluble while the other three are soluble in ethanol. All these drugs can be designed and administered in the sublingual form, i.e. nitroglycerin, procardia and levsin.

6) The use of spray in sublingual nitroglycerin is widely accepted. All the benefits that are listed with sublingual tablets and capsule (may be easily fractured) are true for any sublingual sprays.

7) Sublingual administration includes mixtures of the beneficial medications (statins) as mentioned earlier with nitroglycerin and/or other vasodilators together or individually and platelet aggregation inhibitors or their analogues as determined by the patient's tolerance and/or allergies.

8) Patients in extremis or compromised states can receive the benefits of the statin sublingual administration. A one time or abbreviated use of statins has not been associated with adverse reactions.

9) Nemoprotection will occur in cases of impending strokes and/or strokes.

10) Reduces the size of infarcts.

11) Most importantly, and not shown in prior art as it relates to statin medications and/or combinations, is the fact that absorption through the sublingual entrance into the blood stream does not produce undesirable chemical alterations of the statins and their combinations that will occur with effects as seen in the digestive process of the gastrointestinal tract. This effect is critical and essential with the administration of the high-density lipoproteins.

12) This early emergency administration is crucial for the cardioprotective effect as seen in angina, unstable angina, impending myocardial infarctions (MI), myocardial infarctions, and reinfarctions of the myocardium.

### Examples of Sublingual Drug Characteristics and Ingredients

| | Nitroglycerin | Procardia | Levsin |
|---|---|---|---|
| Water Soluble | + | + | + |
| Alcohol Soluble | + | + | + |
| Non-Volatile Fixing | + | + | + |
| Agent-Polyethylene | | | |
| Glycol 3350 | | | |
| Lactose | + | | |
| Sucrose | + | | |
| Glycerin | + | + | |
| Saccharin Sodium | | + | |
| Peppermint Oil | | + | |
| Dextrates | | | + |
| Colloidal Silicon | | | + |
| Dioxide | | | |
| (water from blood) | | | |
| Mannitol in juices | | | + |
| Stearic Acid | | | + |

### DETAILED DESCRIPTION OF THE INVENTION

In the present invention, there can be an example of a preparation for water-soluble “statins” that may be presented as a tablet dissolving readily with the saliva for sublingual administration. The ingredients that make the conventional nitroglycerin tablet dissolve within one to three minutes are commonly polyethylene glycol 3350, lactose, sucrose and glycerin. Other equivalent substances could be used as well. “Statins” that are not soluble in water but are dissolved in ethanol can be utilized in a gelatin capsule with polyethylene 3350, glycerin, and saccharin and peppermint oil. These capsules should be designed for breakage and immediate release. Finally, the non water-soluble and the water-soluble “statins” can be in liquefied form to be used in an aerosol spray that would allow nitroglycerin to be mixed in appropriate dosages that could be rapidly administered or they may be separate solutions and aerosols as desired.

In the U.S. Pat. No. 6,251,852 B1 of Jun. 26, 2001, reference is made to the combined uses of HMG-CoA reductase inhibitors (statins) and platelet aggregation inhibitors. Discussions centered on the oral administration of the statin preparation in conjunction with the intravenous use of the platelet aggregation inhibitors. Therefore, a need exists in the emergency medicine applications for a route of administration that will allow these medications to be administered without requiring professionals such as seen with use of IV medication administration. Sublingual medications can be absorbed rapidly, almost equivalent to the intravenous route, and not be acted upon by digestive tracts chemical and other mechanical tissue interferences. The sublingual passage is the route that allows for this simple self-administration of medication permitting an almost instant absorption without chemical and/or mechanical interferences, directly into the blood stream. As seen with the examples of nitroglycerin, the effects are felt in seconds. Most medications are altered by the powerful chemical and mechanical undesirable interferences of the gastrointestinal effects. As mentioned, the Rouvastatin intravenous effects are tremendously more powerful than the oral administration of statins. The sublingual method is the closest to the IV route and its resultant effects. We are now in the possession of the technology and progress for the immediate synergism of not only inhibitory statins (and their aforementioned group) and platelet aggregate inhibitors (and their aforementioned group) use but also the natural protection and antioxidant effects of the HDL-cholesterol compounds with their aforementioned group, and the cholesterol ester transfer proteins (CEPT) with their aforementioned group that are the pathway accelerator inhibitors in the cyclical equation of the biosynthesis of cholesterol. See Tables I, II and III. The components and derivatives of these agents represent the current state of the medical research to bring forth newer, safer and more effective medications in this rapidly developing field of cardiovascular, neurovascular and general vascular diseases.

The method for treating impending or catastrophic cardiovascular events, neurovascular events, deep venous thrombosis and embolization; myocardial infarctions; and strokes; includes emergency sublingual administration of a therapeutically effective amount of statins or HMG-CoA reductase inhibitors to a mammal. The reductase inhibitor is preferably selected from the group consisting of: atorvastin; fluvastatin; lovastatin; pravastatin; pharmaceutically acceptable salt, ester and lactone forms thereof, combinations thereof, and derivatives thereof.

Also contemplated is a method for treating impending or catastrophic cardiovascular events, neurovascular events, deep venous thrombosis and embolization; myocardial infarctions; and strokes; including emergency sublingual administration of a therapeutically effective amount of niacin or derivatives thereof, to a mammal.

Also contemplated is a method for treating impending or catastrophic cardiovascular events, neurovascular events, deep venous thrombosis and embolization; myocardial infarctions; and strokes; including emergency administration of a therapeutically effective amount of a cardioprotective agent comprising HDL-cholesterol through sublingual, oral, intravenous and intramuscular administration to a mammal. The HDL-cholesterol is synthesized HDL-cholesterol, natural HDL-cholesterol or pharmaceutically acceptable salts, esters, pro-drugs and solvates thereof.

The cardioprotective agent is subparts of HDL-cholesterol and pharmaceutically acceptable salts, esters, pro-drugs and solvates thereof; or HDL-cholesterol analogues and pharmaceutically acceptable salts, esters, pro-drugs and solvates thereof.

Also contemplated is a method for treating impending or catastrophic cardiovascular events, neurovascular events, deep venous thrombosis and embolization; myocardial infarctions; and strokes; comprising emergency administration of a therapeutically effective amount of a cardioprotective agent including cholesterol ester protein transfer (CEPT) pathway accelerator inhibitors for sublingual, oral, intravenous and intramuscular administration to a mammal. The cardioprotective agent is selected from the group consisting of synthesized cholesterol ester protein transfer (CEPT) pathway accelerator inhibitors and pharmaceutically acceptable salts, esters, pro-drugs and solvates thereof. The cardioprotective agent is selected from the group consisting of subparts of cholesterol ester protein transfer (CEPT) pathway accelerator inhibitors; and pharmaceutically acceptable salts, esters, pro-drugs and solvates thereof.

Also contempated is a method for treating impending or catastrophic cardiovascular events, neurovascular events, deep venous thrombosis and embolization; myocardial infarctions; and strokes; including emergency sublingual administration of a therapeutically effective amount of a platelet aggregation inhibitor to a mammal. The platelet aggregation inhibitor is a glycoprotein IIb/IIIa receptor antagonist; a glycoprotein IIb/IIIa antagonist, ticlopidine, clopidogrel, aspirin, dipyridamole, tirofiban, or the pharmaceutically acceptable salts, esters, pro-drugs and solvates thereof.

Also contemplated is a method of treating impending or catastrophic cardiovascular events, neurovascular events and deep venous thrombosis and embolization; myocardial infarctions; and strokes; including emergency administration of a therapeutic effective amount of an HMG-CoA reductase inhibitor in combination with a therapeutically effective amount of a natural cardioprotective agent comprising a natural HDL-cholesterol in a sublingual, oral, intravenous or intramuscular pathway to a mammal. The cardioprotective agent comprises ApoA-I (fraction of HDL-cholesterol) and pharmaceutically acceptable salts, esters, pro-drugs and solvates thereof; ApoA-II (fraction of HDL-cholesterol) and the pharmaceutically acceptable salts, esters, pro-drugs and solvates thereof; or ApoA-I and ApoA-II (HDL-cholesterol fractions) and pharmaceutically acceptable salts, esters, pro-drugs and solvates thereof.

The cardioprotective agent comprises synthesized HDL-cholesterol and pharmaceutically acceptable salts, esters, pro-drugs and solvates thereof; subparts of HDL-cholesterol and pharmaceutically acceptable salts, esters, pro-drugs and solvates thereof. The HMG-CoA reductase inhibitor is selected from the group consisting of atorvastatin, fluvastatin, lovastatin, and pravastatin and pharmaceutically acceptable salt, ester and lactone forms thereof.

Also contemplated is a method of treating impending or catastrophic cardiovascular events, neurovascular events, deep venous thrombosis and embolization; myocardial infarctions; and strokes; including emergency administration of a therapeutic effective amount of an HMG-CoA reductase inhibitor in combination with a therapeutically effective amount of a cardioprotective agent comprising a cholesterol ester protein transfer (CEPT) in a sublingual, oral, intravenous or intramuscular pathways to a mammal. The cardioprotective agent comprises synthesized cholesterol ester protein transfer (CEPT) pathway accelerator inhibitor; and pharmaceutically acceptable salts, esters, pro-drugs and solvates thereof; subparts of cholesterol ester protein transfer (CEPT) pathway accelerator inhibitors; and pharmaceutically acceptable salts, esters, pro-drugs and solvates thereof; and a derivation of cholesterol ester protein transfer (CEPT) pathway accelerator inhibitors; and pharmaceutically acceptable salts, esters, pro-drugs and solvates thereof.

The HMG-CoA reductase inhibitor is selected from the group consisting of atorvastatin, fluvastatin, lovastatin, pravastatin and pharmaceutically acceptable salt, ester and lactone forms thereof. The HMG-CoA reductase inhibitor is also selected from the group consisting of derivations of atorvastin, fluvastatin, lovastatin and pravastatin and other HMG-CoA reductase inhibitors and pharmaceutically acceptable salt, ester and lactone forms thereof.

Also contemplated is a method of treating impending or catastrophic cardiovascular events, neurovascular events, deep venous thrombosis and embolization; myocardial infarctions; and strokes; including emergency sublingual administration of therapeutic effective amount of an HMG-CoA reductase inhibitor in combination with a cardioprotective agent including a therapeutically effective amount of niacin to a mammal. The cardioprotective agent is selected from an effective amount of a niacin analogues for sublingual administration to a mammal and pharmaceutically acceptable salts, esters, pro-drug and lactone forms thereof. The HMG-CoA reductase inhibitor is selected from the group consisting of atorvastatin, fluvastatin, lovastatin, pravastatin and pharmaceutically acceptable salts, esters, and lactone forms thereof. The HMG-CoA reductase inhibitor is also selected from the group consisting of derivations of atorvastatin, fluvastatin, lovastatin, pravastatin and pharmaceutically acceptable salt, ester and lactone forms thereof.

Also contemplated is a method of treating impending catastrophic cardiovascular events, neurovascular events, deep venous thrombosis and embolization; myocardial infarctions; and strokes; comprising emergency sublingual administration of therapeutic effective amount of an HMG-CoA reductase inhibitor in combination with a therapeutically effective amount of a platelet aggregation inhibitor to a mammal. The HMG-CoA reductase inhibitor is selected from the group consisting of atorvastatin, fluvastatin, lovastatin, pravastatin and pharmaceutically acceptable salts, esters, and lactone forms thereof. The HMG-CoA reductase inhibitor is also selected from the group consisting of derivations of atorvastatin, fluvastatin, lovastatin, pravastatin and pharmaceutically acceptable salt, ester and lactone forms thereof. The platelet aggregation inhibitor is selected from the group consisting of a glycoprotein IIb/IIIa antagonist, ticlopidine, clopidogrel, aspirin, dipyridamole and their derivations and pharmaceutically acceptable salts, esters, and lactone forms thereof. The recognized platelet aggregation inhibitor is also selected from tirofiban and pharmaceutically acceptable salts, esters, pro-drugs and solvates thereof.

Also contemplated is a method for treating impending or catastrophic cardiovascular events, neurovascular events, deep venous thrombosis and embolization; myocardial infarctions; and strokes; including emergency administration of a therapeutically effective amount of an HMG-CoA reductase inhibitor in combination with a therapeutically effective amount of cholesterol ester protein transfer (CEPT) pathway accelerator inhibitors and niacin in a sublingual, oral, intravenous and intramuscular administration to a mammal. The HMG-CoA reductase inhibitor is selected from the group consisting of atorvastatin, fluvastatin, lovastatin, pravastatin and pharmaceutically acceptable salts, esters, and lactone forms thereof. The HMG-CoA reductase inhibitor is also selected from the group consisting of derivations of atorvastatin, fluvastatin, lovastatin, pravastatin and pharmaceutically acceptable salt, ester and lactone forms thereof. The niacin is selected from the group consisting of niacin analogues and pharmaceutically acceptable salts, esters, pro-drugs and solvates thereof. The cholesterol ester protein transfer (CEPT) pathway accelerator inhibitors is selected from the group consisting of natural (CEPT), synthetic (CEPT), subparts of (CEPT) analogues of (CEPT) and pharmaceutically acceptable salts, esters, pro-drugs and solvates thereof.

Also contemplated is a method for treating impending or catastrophic cardiovascular events, neurovascular events, deep venous thrombosis and embolization; myocardial infarctions; and strokes; comprising emergency administration of a therapeutically effective amount of an HMG-CoA reductase inhibitor in combination with a therapeutically effective amount a cholesterol ester protein transfer (CEPT) pathway accelerator and a platelet aggregation inhibitor in a sublingual, oral, intravenous and intramuscular administration to a mammal. The HMG-CoA reductase inhibitor is selected from the group comprising atorvastatin, fluvastatin, lovastatin, pravastatin and pharmaceutically acceptable salt, ester and lactone forms thereof, and derivatives thereof. The therapeutically effective cholesterol ester protein transfer (CEPT) pathway accelerator inhibitors is selected from the group consisting of natural (CEPT), synthetic (CEPT), subparts of (CEPT) analogues of (CEPT) and pharmaceutically acceptable salts, esters, pro-drugs and solvates thereof. The platelet aggregation inhibitor is a glycoprotein IIb/IIIa receptor antagonist, glycoprotein IIb/IIIa antagonist, ticlopidine, clopidogrel, aspirin, dipyridamole, tirofiban or pharmaceutically acceptable salts, esters, pro-drugs and solvates thereof.

Also contemplated method for treating impending or catastrophic cardiovascular events, neurovascular events, deep venous thrombosis and embolization; myocardial infarctions; and strokes; including emergency administration of a therapeutically effective amount of an HMG-CoA reductase inhibitor in combination with a therapeutically effective amount of cholesterol ester protein transfer (CEPT) pathway accelerator inhibitor and HDL-cholesterol in a sublingual, oral, intravenous and intramuscular administration to a mammal. The HMG-CoA reductase inhibitor is selected from the group comprising atorvastatin, fluvastatin, lovastatin, pravastatin and pharmaceutically acceptable salt, ester and lactone forms thereof. The HDL-cholesterol is selected from the group consisting of ApoA-I and pharmaceutically acceptable salts, esters, pro-drugs and solvates thereof, ApoA-II and pharmaceutically acceptable salts, esters, pro-drugs and solvates thereof, and mixtures thereof. The HDL-cholesterol is also synthesized HDL-cholesterol and pharmaceutically acceptable salts, esters, pro-drugs and solvates thereof, HDL-cholesterol subparts and pharmaceutically acceptable salts, esters, pro-drugs and solvates thereof, or HDL-cholesterol analogues and pharmaceutically acceptable salts, esters, pro-drugs and solvates thereof. The therapeutically effective cholesterol ester protein transfer (CEPT) pathway accelerator inhibitor is selected from the group consisting of natural (CEPT), synthetic (CEPT), subparts of (CEPT), analogues of (CEPT) and pharmaceutically acceptable salts, esters, pro-drugs and solvates thereof.

Also contemplated is a method for treating impending or catastrophic cardiovascular events, neurovascular events, deep venous thrombosis and embolization; myocardial infarctions; and strokes; including emergency administration of a therapeutically effective amount of an HMG-CoA reductase inhibitor in combination with a therapeutically effective amount of natural HDL-cholesterol and niacin in a sublingual, oral, intravenous and intramuscular administration to a mammal. The HMG-CoA reductase inhibitor is selected from the group consisting of atorvastatin, fluvastatin, lovastatin, pravastatin and pharmaceutically acceptable salt, ester and lactone forms thereof, and derivatives thereof. The HDL-cholesterol is selected from the group consisting of ApoA-I and pharmaceutically acceptable salts, esters, pro-drugs and solvates thereof, ApoA-II and pharmaceutically acceptable salts, esters, pro-drugs and solvates thereof, and mixtures thereof. The cardioprotective agent is also a synthesized HDL-cholesterol, a subpart of HDL-cholesterol, HDL-cholesterol analogues and pharmaceutically acceptable salts, esters, pro-drugs and solvates thereof. The niacin is niacin analogues and pharmaceutically acceptable salts, esters, pro-drugs and lactone forms thereof.

Also contemplated is a method for treating impending or catastrophic cardiovascular events, neurovascular events, deep venous thrombosis and embolization; myocardial infarctions; and strokes; including emergency administration of a therapeutically effective amount of an HMG-CoA reductase inhibitor in combination with a therapeutically effective amount a natural HDL-cholesterol and a platelet aggregation inhibitor in a sublingual, oral, intravenous and intramuscular administration to a mammal. The HMG-CoA reductase inhibitor is selected from the group consisting of atorvastatin, fluvastatin, lovastatin, pravastatin and pharmaceutically acceptable salt, ester and lactone forms thereof, and derivatives thereof. The HDL-cholesterol is selected from the group consisting of ApoA-I and pharmaceutically acceptable salt, esters, pro-drugs and solvates thereof, ApoA-II and the pharmaceutically acceptable salts, esters, pro-drugs and solvates thereof, and mixtures thereof. The cardioprotective agent includes a synthesized HDL-cholesterol, a subpart of HDL-cholesterol, HDL-cholesterol analogues and pharmaceutically acceptable salts, esters, pro-drugs and solvates thereof. The platelet aggregation inhibitor is a glycoprotein IIb/IIIa receptor antagonist, glycoprotein IIb/IIIa antagonist, ticlopidine, clopidogrel, aspirin, dipyridamole, tirofiban and pharmaceutically acceptable salts, esters, pro-drugs and solvates thereof.

Also contemplated is a method for treating impending or catastrophic cardiovascular events, neurovascular events, deep venous thrombosis and embolization; myocardial infarctions; and strokes; including emergency administration of a therapeutically effective amount of cholesterol ester protein transfer (CEPT) pathway accelerator inhibitors in combination with a therapeutically effective amount of niacin in a sublingual, oral, intravenous and intramuscular administration to a mammal. The therapeutically effective cholesterol ester protein transfer (CEPT) pathway accelerator inhibitor is a natural (CEPT), synthetic (CEPT), subparts of (CEPT) or analogues of (CEPT) and pharmaceutically acceptable salts, esters, pro-drugs and solvates thereof. The niacin is a niacin analogues and pharmaceutically acceptable salts, esters, pro-drugs and lactone forms thereof.

Also contemplated is a method for treating impending or catastrophic cardiovascular events, neurovascular events, deep venous thrombosis and embolization; myocardial infarctions; and strokes; including emergency administration of a therapeutically effective amount of cholesterol ester protein transfer (CEPT) pathway accelerator and a therapeutically effective amount of a platelet aggregation inhibitor in a sublingual, oral, intravenous and intramuscular administration to a mammal. The pharmaceutically effective cholesterol ester protein transfer (CEPT) pathway accelerator inhibitor is natural (CEPT), synthetic (CEPT), subparts of (CEPT) or analogues of (CEPT) and pharmaceutically acceptable salts, esters, pro-drugs and solvates thereof. The platelet aggregation inhibitor is a glycoprotein IIb/IIIa receptor antagonist, a glycoprotein IIb/IIIa antagonist, ticlopidine, clopidogrel, aspirin, dipyridamole, or tirofiban and the pharmaceutically acceptable salts, esters, pro-drugs and solvates thereof.

Also contemplated is a method for treating impending or catastrophic cardiovascular events, neurovascular events, deep venous thrombosis and embolization; myocardial infarctions; and strokes; including emergency administration of a therapeutically effective amount of a therapeutically effective amount of cholesterol ester protein transfer (CEPT) pathway accelerator inhibitor and a therapeutically effective amount of the HDL-cholesterol in a sublingual, oral, intravenous and intramuscular administration to a mammal. The therapeutically effective cholesterol ester protein transfer (CEPT) pathway accelerator inhibitor is natural (CEPT), synthetic (CEPT), subparts of (CEPT) or analogues of (CEPT) and pharmaceutically acceptable salts, esters, pro-drugs and solvates thereof. The HDL-cholesterol is synthesized HDL-cholesterol and pharmaceutically acceptable salts, esters, pro-drugs and solvates thereof, subparts of HDL-cholesterol and pharmaceutically acceptable salts, esters, pro-drugs and solvates thereof, or HDL-cholesterol analogues and the pharmaceutically acceptable salts, esters, pro-drugs and solvates thereof.

Also contemplated is a method of treating impending or catastrophic cardiovascular event, neurovascular event, deep venous thrombosis and embolization; myocardial infarctions; and strokes; including emergency administration of therapeutic effective amount of an HMG-CoA reductase inhibitor in combination with a therapeutically effective amount of nitroglycerin in a sublingual or intravenous pathway to a mammal. The HMG-CoA reductase inhibitor is selected from the group comprising derivations of atorvastatin, fluvastatin, lovastatin, pravastatin and the pharmaceutically acceptable salt, ester and lactone forms thereof.

What is claimed is:

1. A method for treating impending or catastrophic cardiovascular events, neurovascular events, deep venous thrombosis and embolization; myocardial infarctions; and strokes; comprising emergency sublingual administration of a therapeutically effective amount of statins or HMG-CoA reductase inhibitors to a mammal.

2. The method of claim 1, wherein the HMG-CoA reductase inhibitor is selected from the group consisting of: atorvastin; fluvastatin; lovastatin; pravastatin; pharmaceutically acceptable salt, ester and lactone forms thereof, combinations thereof, and derivatives thereof.

3. A method for treating impending or catastrophic cardiovascular events, neurovascular events, deep venous thrombosis and embolization; myocardial infarctions; and strokes; comprising emergency sublingual administration of a therapeutically effective amount of niacin or derivatives thereof, to a mammal.

4. A method for treating impending or catastrophic cardiovascular events, neurovascular events, deep venous thrombosis and embolization; myocardial infarctions; and strokes; comprising emergency administration of a therapeutically effective amount of a cardioprotective agent comprising HDL-cholesterol through sublingual, oral, intravenous and intramuscular administration to a mammal.

5. The method set forth in claim 4, wherein the HDL-cholesterol is synthesized HDL-cholesterol, natural HDL-cholesterol or pharmaceutically acceptable salts, esters, pro-drugs and solvates thereof.

6. The method set forth in claim 4, wherein the cardioprotective agent is subparts of HDL-cholesterol or pharmaceutically acceptable salts, esters, pro-drugs and solvates thereof.

7. The method set forth in claim 4, wherein the cardioprotective agent is selected from the group consisting of HDL-cholesterol analogues and pharmaceutically acceptable salts, esters, pro-drugs and solvates thereof.

8. A method for treating impending or catastrophic cardiovascular events, neurovascular events, deep venous thrombosis and embolization; myocardial infarctions; and strokes; comprising emergency administration of a therapeutically effective amount of a cardioprotective agent comprising cholesterol ester protein transfer (CEPT) pathway accelerator inhibitors for sublingual, oral, intravenous and intramuscular administration to a mammal.

9. The method set forth in claim 8, wherein the cardioprotective agent is selected from the group consisting of synthesized cholesterol ester protein transfer (CEPT) pathway accelerator inhibitors and pharmaceutically acceptable salts, esters, pro-drugs and solvates thereof.

10. The method set forth in claim 8, wherein the cardioprotective agent is selected from the group consisting of subparts of cholesterol ester protein transfer (CEPT) pathway accelerator inhibitors; and pharmaceutically acceptable salts, esters, pro-drugs and solvates thereof.

11. A method for treating impending or catastrophic cardiovascular events, neurovascular events, deep venous thrombosis and embolization; myocardial infarctions; and strokes; comprising emergency sublingual administration of a therapeutically effective amount of a platelet aggregation inhibitor to a mammal.

12. The method set forth in claim 11, wherein the platelet aggregation inhibitor is a glycoprotein IIb/IIIa receptor antagonist.

13. The method set forth in claim 11, wherein the platelet aggregation inhibitor is selected from the group consisting of a glycoprotein IIb/IIIa antagonist, ticlopidine, clopidogrel, aspirin and dipyridamole.

14. The method set forth in claim 11, wherein the platelet aggregation inhibitor is tirofiban and the pharmaceutically acceptable salts, esters, pro-drugs and solvates thereof.

15. A method of treating impending or catastrophic cardiovascular events, neurovascular events and deep venous thrombosis and embolization; myocardial infarctions; and strokes; comprising emergency administration of a therapeutic effective amount of an HMG-CoA reductase inhibitor in combination with a therapeutically effective amount of a natural cardioprotective agent comprising a natural HDL-cholesterol in a sublingual, oral, intravenous or intramuscular pathway to a mammal.

16. The method set forth in claim 15, wherein the cardioprotective agent comprises ApoA-I (fraction of HDL-cholesterol) and pharmaceutically acceptable salts, esters, pro-drugs and solvates thereof.

17. The method set forth in claim 15, wherein the cardioprotective agent comprises ApoA-II (fraction of HDL-cholesterol) and the pharmaceutically acceptable salts, esters, pro-drugs and solvates thereof.

18. The method set forth in claim 15, wherein the cardioprotective agent comprises ApoA-I and ApoA-II (HDL-cholesterol fractions) and pharmaceutically acceptable salts, esters, pro-drugs and solvates thereof.

19. The method set forth in claim 15, wherein the cardioprotective agent comprises synthesized HDL-cholesterol and pharmaceutically acceptable salts, esters, pro-drugs and solvates thereof.

20. The method set forth in claim 15, wherein the cardioprotective agent comprises subparts of HDL-cholesterol and pharmaceutically acceptable salts, esters, pro-drugs and solvates thereof.

21. The method set forth in claim 15, wherein the HMG-CoA reductase inhibitor is selected from the group consisting of atorvastatin, fluvastatin, lovastatin, and pravastatin and pharmaceutically acceptable salt, ester and lactone forms thereof.

23. A method of treating impending or catastrophic cardiovascular events, neurovascular events, deep venous thrombosis and embolization; myocardial infarctions; and strokes; comprising emergency administration of a therapeutic effective amount of an HMG-CoA reductase inhibitor in combination with a therapeutically effective amount of a cardioprotective agent comprising a cholesterol ester protein transfer (CEPT) in a sublingual, oral, intravenous or intramuscular pathways to a mammal.

24. The method set forth in claim 23, wherein the cardioprotective agent comprises synthesized cholesterol ester protein transfer (CEPT) pathway accelerator inhibitor; and pharmaceutically acceptable salts, esters, pro-drugs and solvates thereof.

25. The method set forth in claim 23, wherein the cardioprotective agent comprises subparts of cholesterol ester protein transfer (CEPT) pathway accelerator inhibitors; and pharmaceutically acceptable salts, esters, pro-drugs and solvates thereof.

26. The method set forth in claim 23, wherein the cardioprotective agent comprises a derivation of cholesterol ester protein transfer (CEPT) pathway accelerator inhibitors; and pharmaceutically acceptable salts, esters, pro-drugs and solvates thereof.

27. The method set forth in claim 23, wherein the HMG-CoA reductase inhibitor is selected from the group consisting of atorvastatin, fluvastatin, lovastatin, pravastatin and pharmaceutically acceptable salt, ester and lactone forms thereof.

28. The method of claim 23, wherein the HMG-CoA reductase inhibitor is selected from the group consisting of derivations of atorvastin, fluvastatin, lovastatin and pravastatin and other HMG-CoA reductase inhibitors and pharmaceutically acceptable salt, ester and lactone forms thereof.

29. A method of treating impending or catastrophic cardiovascular events, neurovascular events, deep venous thrombosis and embolization; myocardial infarctions; and strokes; comprising emergency sublingual administration of therapeutic effective amount of an HMG-CoA reductase inhibitor in combination with a cardioprotective agent comprising a therapeutically effective amount of niacin to a mammal.

30. The method set forth in claim 29, wherein the cardioprotective agent is selected from an effective amount of a niacin analogues for sublingual administration to a mammal and pharmaceutically acceptable salts, esters, pro-drug and lactone forms thereof.

31. The method set forth in claim 29, wherein the HMG-CoA reductase inhibitor is selected from the group consisting of atorvastatin, fluvastatin, lovastatin, pravastatin and pharmaceutically acceptable salts, esters, and lactone forms thereof.

32. The method set forth in claim 29, wherein the HMG-CoA reductase inhibitor is selected from the group consisting of derivations of atorvastatin, fluvastatin, lovastatin, pravastatin and pharmaceutically acceptable salt, ester and lactone forms thereof.

33. A method of treating impending catastrophic cardiovascular events, neurovascular events, deep venous thrombosis and embolization; myocardial infarctions; and strokes; comprising emergency sublingual administration of therapeutic effective amount of an HMG-CoA reductase inhibitor in combination with a therapeutically effective amount of a platelet aggregation inhibitor to a mammal.

34. The method set forth in claim 33, wherein the HMG-CoA reductase inhibitor is selected from the group consisting of atorvastatin, fluvastatin, lovastatin, pravastatin and pharmaceutically acceptable salts, esters, and lactone forms thereof.

35. The method of claim 33, wherein the HMG-CoA reductase inhibitor is selected from the group consisting of derivations of atorvastatin, fluvastatin, lovastatin, pravastatin and pharmaceutically acceptable salt, ester and lactone forms thereof.

36. The method set forth in claim 33, wherein the platelet aggregation inhibitor is selected from the group consisting of a glycoprotein IIb/IIIa antagonist, ticlopidine, clopidogrel, aspirin, dipyridamole and their derivations and pharmaceutically acceptable salts, esters, and lactone forms thereof.

37. The method set forth in claim 33, wherein the platelet aggregation inhibitor is selected from tirofiban and pharmaceutically acceptable salts, esters, pro-drugs and solvates thereof.

38. A method for treating impending or catastrophic cardiovascular events, neurovascular events, deep venous thrombosis and embolization; myocardial infarctions; and strokes; comprising emergency administration of a therapeutically effective amount of an HMG-CoA reductase inhibitor in combination with a therapeutically effective amount of cholesterol ester protein transfer (CEPT) pathway accelerator inhibitors and niacin in a sublingual, oral, intravenous and intramuscular administration to a mammal.

39. The method set forth in claim 38, wherein the HMG-CoA reductase inhibitor is selected from the group consisting of atorvastatin, fluvastatin, lovastatin, pravastatin and pharmaceutically acceptable salts, esters, and lactone forms thereof.

40. The method of claim 38, wherein the HMG-CoA reductase inhibitor is selected from the group consisting of derivations of atorvastatin, fluvastatin, lovastatin, pravastatin and pharmaceutically acceptable salt, ester and lactone forms thereof.

41. The method set forth in claim 38, wherein the niacin is selected from the group consisting of niacin analogues and pharmaceutically acceptable salts, esters, pro-drugs and solvates thereof.

42. The method set forth in claim 38, wherein the cholesterol ester protein transfer (CEPT) pathway accelerator inhibitors is selected from the group consisting of natural (CEPT), synthetic (CEPT), subparts of (CEPT) analogues of (CEPT) and pharmaceutically acceptable salts, esters, pro-drugs and solvates thereof.

43. A method for treating impending or catastrophic cardiovascular events, neurovascular events, deep venous thrombosis and embolization; myocardial infarctions; and strokes; comprising emergency administration of a therapeutically effective amount of an HMG-CoA reductase inhibitor in combination with a therapeutically effective amount a cholesterol ester protein transfer (CEPT) pathway accelerator and a platelet aggregation inhibitor in a sublingual, oral, intravenous and intramuscular administration to a mammal.

44. The method set forth in claim 43, wherein the HMG-CoA reductase inhibitor is selected from the group comprising atorvastatin, fluvastatin, lovastatin, pravastatin and pharmaceutically acceptable salt, ester and lactone forms thereof, and derivatives thereof.

45. The method set forth in claim 43, wherein the therapeutically effective cholesterol ester protein transfer (CEPT) pathway accelerator inhibitors is selected from the group consisting of natural (CEPT), synthetic (CEPT), subparts of (CEPT) analogues of (CEPT) and pharmaceutically acceptable salts, esters, pro-drugs and solvates thereof.

46. The method set forth in claim 43, wherein the platelet aggregation inhibitor is a glycoprotein IIb/IIIa receptor antagonist, glycoprotein IIb/IIIa antagonist, ticlopidine, clopidogrel, aspirin, dipyridamole, tirofiban or pharmaceutically acceptable salts, esters, pro-drugs and solvates thereof.

47. A method for treating impending or catastrophic cardiovascular events, neurovascular events, deep venous thrombosis and embolization; myocardial infarctions; and strokes; comprising emergency administration of a therapeutically effective amount of an HMG-CoA reductase inhibitor in combination with a therapeutically effective amount of cholesterol ester protein transfer (CEPT) pathway accelerator inhibitor and HDL-cholesterol in a sublingual, oral, intravenous and intramuscular administration to a mammal.

48. The method set forth in claim 47, wherein the HMG-CoA reductase inhibitor is selected from the group comprising atorvastatin, fluvastatin, lovastatin, pravastatin and pharmaceutically acceptable salt, ester and lactone forms thereof.

49. The method set forth in claim 47, wherein the HDL-cholesterol is selected from the group consisting of ApoA-I and pharmaceutically acceptable salts, esters, pro-drugs and solvates thereof, ApoA-II and pharmaceutically acceptable salts, esters, pro-drugs and solvates thereof, and mixtures thereof.

50. The method set forth in claim 47, wherein the HDL-cholesterol is synthesized HDL-cholesterol and pharmaceutically acceptable salts, esters, pro-drugs and solvates thereof, HDL-cholesterol subparts and pharmaceutically acceptable salts, esters, pro-drugs and solvates thereof, or HDL-cholesterol analogues and pharmaceutically acceptable salts, esters, pro-drugs and solvates thereof.

51. The method set forth in claim 47, wherein the therapeutically effective cholesterol ester protein transfer (CEPT) pathway accelerator inhibitor is selected from the group consisting of natural (CEPT), synthetic (CEPT), subparts of (CEPT), analogues of (CEPT) and pharmaceutically acceptable salts, esters, pro-drugs and solvates thereof.

52. A method for treating impending or catastrophic cardiovascular events, neurovascular events, deep venous thrombosis and embolization; myocardial infarctions; and strokes; comprising emergency administration of a therapeutically effective amount of an HMG-CoA reductase inhibitor in combination with a therapeutically effective amount of natural HDL-cholesterol and niacin in a sublingual, oral, intravenous and intramuscular administration to a mammal.

53. The method fo claim 52, wherein the HMG-CoA reductase inhibitor is selected from the group consisting of atorvastatin, fluvastatin, lovastatin, pravastatin and pharmaceutically acceptable salt, ester and lactone forms thereof, and derivatives thereof.

54. The method set forth in claim 52, wherein the HDL-cholesterol is selected from the group consisting of ApoA-I and pharmaceutically acceptable salts, esters, pro-drugs and solvates thereof, ApoA-II and pharmaceutically acceptable salts, esters, pro-drugs and solvates thereof, and mixtures thereof.

55. The method set forth in claim 52, wherein the cardioprotective agent is a synthesized HDL-cholesterol, a subpart of HDL-cholesterol, HDL-cholesterol analogues and pharmaceutically acceptable salts, esters, pro-drugs and solvates thereof.

56. The method set forth in claim 52, wherein the niacin is niacin analogues and pharmaceutically acceptable salts, esters, pro-drugs and lactone forms thereof.

57. A method for treating impending or catastrophic cardiovascular events, neurovascular events, deep venous thrombosis and embolization; myocardial infarctions; and strokes; comprising emergency administration of a therapeutically effective amount of an HMG-CoA reductase inhibitor in combination with a therapeutically effective amount a natural HDL-cholesterol and a platelet aggregation inhibitor in a sublingual, oral, intravenous and intramuscular administration to a mammal.

58. The method set forth in claim 57, wherein the HMG-CoA reductase inhibitor is selected from the group consisting of atorvastatin, fluvastatin, lovastatin, pravastatin and pharmaceutically acceptable salt, ester and lactone forms thereof, and derivatives thereof.

59. The method of set forth claim 57 wherein the HDL-cholesterol is selected from the group consisting of ApoA-I and pharmaceutically acceptable salt, esters, pro-drugs and solvates thereof, ApoA-II and the pharmaceutically acceptable salts, esters, pro-drugs and solvates thereof, and mixtures thereof.

60. The method set forth in claim 57, wherein the cardioprotective agent comprises a synthesized HDL-cholesterol, a subpart of HDL-cholesterol, HDL-cholesterol analogues and pharmaceutically acceptable salts, esters, pro-drugs and solvates thereof.

61. The method set forth in claim 57, wherein the platelet aggregation inhibitor is a glycoprotein IIb/IIIa receptor antagonist, glycoprotein IIb/IIIa antagonist, ticlopidine, clopidogrel, aspirin, dipyridamole, tirofiban and pharmaceutically acceptable salts, esters, pro-drugs and solvates thereof.

62. A method for treating impending or catastrophic cardiovascular events, neurovascular events, deep venous thrombosis and embolization; myocardial infarctions; and strokes; comprising emergency administration of a therapeutically effective amount of cholesterol ester protein transfer (CEPT) pathway accelerator inhibitors in combination with a therapeutically effective amount of niacin in a sublingual, oral, intravenous and intramuscular administration to a mammal.

63. The method set forth in claim 62, wherein the therapeutically effective cholesterol ester protein transfer (CEPT) pathway accelerator inhibitor is a natural (CEPT), synthetic (CEPT), subparts of (CEPT) or analogues of (CEPT) and pharmaceutically acceptable salts, esters, pro-drugs and solvates thereof.

64. The method set forth in claim 62, wherein the niacin is a niacin analogues and pharmaceutically acceptable salts, esters, pro-drugs and lactone forms thereof.

65. A method for treating impending or catastrophic cardiovascular events, neurovascular events, deep venous thrombosis and embolization; myocardial infarctions; and strokes; comprising emergency administration of a therapeutically effective amount of cholesterol ester protein transfer (CEPT) pathway accelerator and a therapeutically effective amount of a platelet aggregation inhibitor in a sublingual, oral, intravenous and intramuscular administration to a mammal.

66. The method set forth in claim 54, wherein the pharmaceutically effective cholesterol ester protein transfer (CEPT) pathway accelerator inhibitor is natural (CEPT), synthetic (CEPT), subparts of (CEPT) or analogues of (CEPT) and pharmaceutically acceptable salts, esters, pro-drugs and solvates thereof.

67. The method set forth in claim 65, wherein the platelet aggregation inhibitor is a glycoprotein IIb/IIIa receptor antagonist, a glycoprotein IIb/IIIa antagonist, ticlopidine, clopidogrel, aspirin, dipyridamole, or tirofiban and the pharmaceutically acceptable salts, esters, pro-drugs and solvates thereof.

68. A method for treating impending or catastrophic cardiovascular events, neurovascular events, deep venous thrombosis and embolization; myocardial infarctions; and strokes; comprising emergency administration of a therapeutically effective amount of a therapeutically effective amount of cholesterol ester protein transfer (CEPT) pathway accelerator inhibitor and a therapeutically effective amount of the HDL-cholesterol in a sublingual, oral, intravenous and intramuscular administration to a mammal.

69. The method set forth in claim 68 wherein the therapeutically effective cholesterol ester protein transfer (CEPT) pathway accelerator inhibitor is natural (CEPT), synthetic (CEPT), subparts of (CEPT) or analogues of (CEPT) and pharmaceutically acceptable salts, esters, pro-drugs and solvates thereof.

70. The method of claim 69, wherein the HDL-cholesterol is synthesized HDL-cholesterol and pharmaceutically acceptable salts, esters, pro-drugs and solvates thereof, subparts of HDL-cholesterol and pharmaceutically acceptable salts, esters, pro-drugs and solvates thereof, or HDL-cholesterol analogues and the pharmaceutically acceptable salts, esters, pro-drugs and solvates thereof.

71. A method of treating impending or catastrophic cardiovascular event, neurovascular event, deep venous thrombosis and embolization; myocardial infarctions; and strokes; comprising of emergency administration of therapeutic effective amount of an HMG-CoA reductase inhibitor in combination with a therapeutically effective amount of nitroglycerin in a sublingual or intravenous pathway to a mammal.

72. The method set forth in claim 71, wherein the HMG-CoA reductase inhibitor is selected from the group comprising derivations of atorvastatin, fluvastatin, lovastatin, pravastatin and the pharmaceutically acceptable salt, ester and lactone forms thereof.
